(19) United States
(12) Plant Patent Application Publication
(10) Pub. No.: US 2019/0059184 P1
(43) Pub. Date: Feb. 21, 2019

(54) Mango plant named `NMBP-4069'

(50) ;

(71) Applicant: Commonwealth Scientific and Industrial Research Organisation, Acton Act (AU)

(72) Inventor: Ian Stephen Edward BALLY, Mareeba (AU)

(73) Assignee: Commonwealth Scientific and Industrial Research Organisation, Acton Act (AU)

(21) Appl. No.: 15/731,842

(22) Filed: Aug. 15, 2017

(51) Int. Cl. A01H 6/00

(52) U.S. Cl. PLT/159

(57) ABSTRACT

A new and distinct variety of Mango plant, herein referred to by its cultivar name, ‘NMBP-4069’, is provided which forms fruit with an average weight of 378 grams and with skin that is soft pink to red blush over a pale yellow background in coloration. The new variety is a mid-season variety.

Botanical/commercial classification: Latin name—Mangifera indica L. Common name—Mango Plant. Varietal Denomination—‘NMBP-4069’.

### SUMMARY OF THE INVENTION

The new variety of Mangifera indica Mango plant of the present invention was created by closed pollination using hand pollination techniques in 1994 in Northern Territory, Australia wherein two parents which previously had been studied were crossed with the intent that they would contribute the desired characteristics. The new cultivar was selected after comparative evaluation of tree and fruit characteristics over several seasons at two sites: Mareeba, Queensland and Darwin, Northern Territory in Australia. The female parent (i.e., seed parent) of the new variety was the ‘Van Dyke’ variety (non-patented). The male parent (i.e., pollen parent) was the ‘Kensington Pride’ variety (non-patented).

The parentage can be summarized as follows:

‘Van Dyke’x‘Kensington Pride’.

The seeds resulting from the above pollination were sown and small plants were obtained which were physically and biologically different from each other. The new variety was grafted on the ‘Kensington Pride’ variety in late 1995 and field planted at Southedge research station, Queensland, Australia on Mar. 4, 1996. The candidate cultivar was selected after comparative evaluation of tree and fruit characteristics over several seasons. Selective study resulted in the identification of a single plant of the new variety.

It was found that the new Mango plant of the present invention possesses the following combination of characteristics:
(a) displays a medium vigor plant with a compact dense canopy,
(b) exhibits a medium to heavy yielding with a tendency for biennial bearing,
(c) produces monoembryonic seed,
(d) has a harvest season that is early to mid-season, and
(e) provides a fruit with a sweet rich ‘Kensington Pride’ variety style flavor with a slight tang.

The new variety successfully meets the needs of the horticultural industry.

The new variety can be readily distinguished from its ancestors. More specifically, the ‘Van Dyke’ variety provides mature fruit that has a flattened shape at the stylar scar and has a Floridian type flavor, and the ‘Kensington Pride’ variety provides a concave young leaf shape in cross section, whereas the new variety provides mature fruit that has a pointed shape at the stylar scar, has a sweet rich ‘Kensington Pride’ variety style with a slight tang flavor, and provides a straight young leaf shape in cross section. Additionally, the new variety can be readily distinguished from other known cultivars. For example, the ‘Delta R2E2’ variety (non-patented) produces polyembryonic seeds, while the new variety is monoembryonic.

The new variety has been found to undergo asexual propagation in Australia by a number of routes, including by grafting of cuttings. The typical asexual propagation of the new variety has been to initially increase the numbers of trees for advanced trials and evaluation and more recently for supply of on-farm large scale trials of the variety. Propagation has been by Whip and Tongue or cleft grafting of the new variety (scion) on to a seedling rootstock of the variety ‘Kensington Pride’ in the Mareeba district of Queensland, Darwin in the Northern Territory, or Kununurra in Western Australia. Asexual propagation in Australia has shown that the characteristics of the new variety are stable and strictly transmissible by asexual propagation from one generation to another. Accordingly, the new variety undergoes asexual propagation in a true-to-type manner.

The new variety has been named ‘NMBP-4069’.

### BRIEF DESCRIPTION OF THE PHOTOGRAPHS

The accompanying photographs show, as nearly true as it is reasonably possible to make the same in color illustrations of this character, typical specimens of the new variety.

FIG. 1—illustrates specimens of multiple branches with fruit on an eight year-old tree.

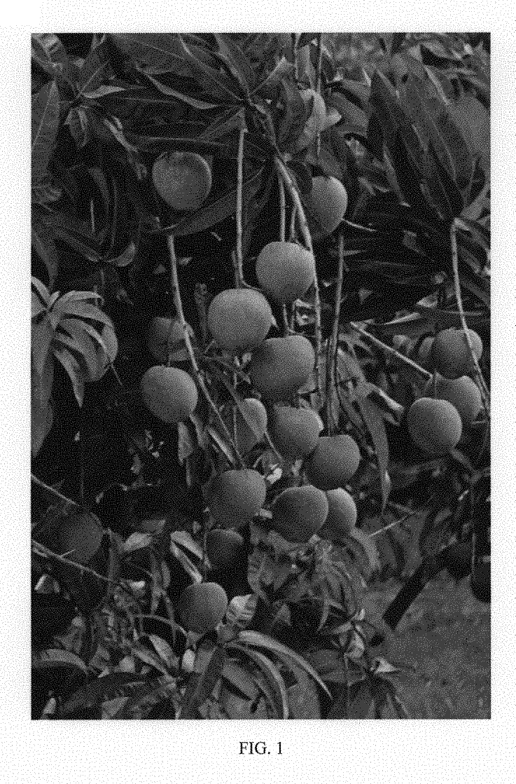

FIG. 2—illustrates a specimen of a mature leaf—upper surface.

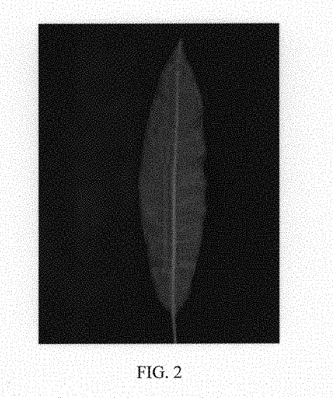

FIG. 3—illustrates a specimen of an immature leaf—lower surface.

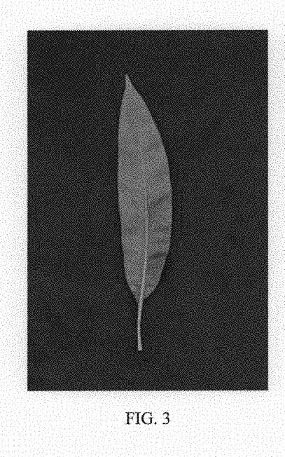

FIG. 4—illustrates a specimen of a single three year-old plant with flowers.

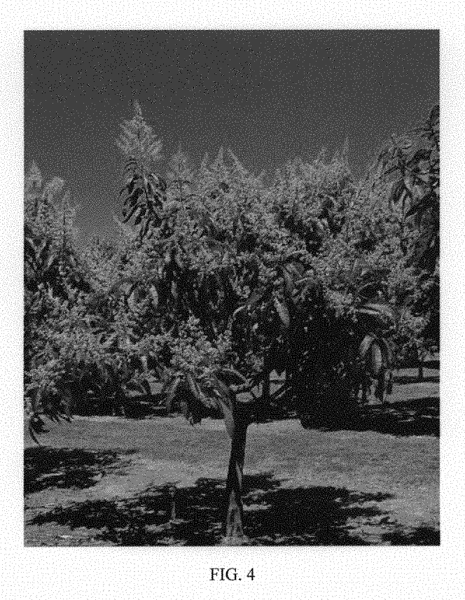

FIG. 5—illustrates a specimen of a single six year-old plant with mature fruit.

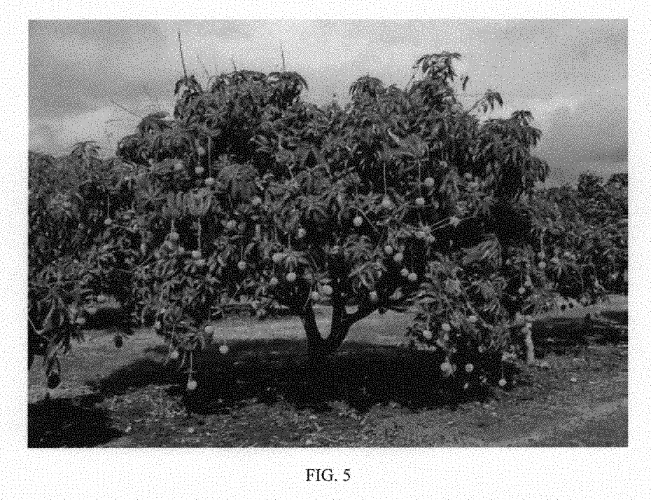

FIG. 6—illustrates specimens of multiple fruit growing on an inflorescence on a six year-old tree.

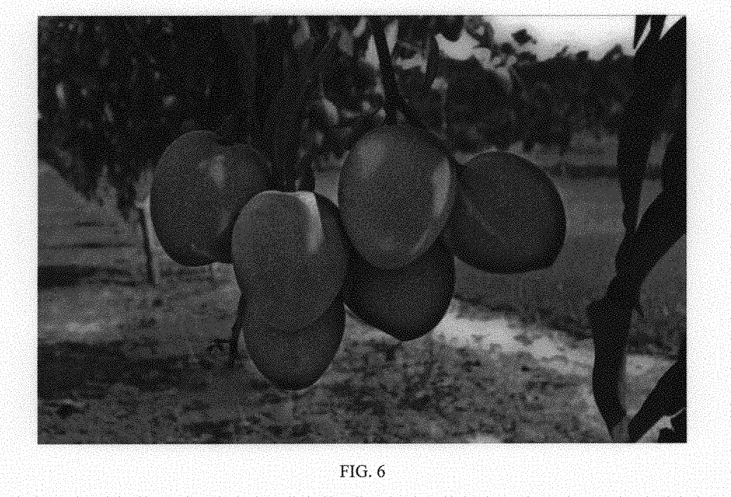

FIG. 7—illustrates specimens of multiple plants with mature fruit.

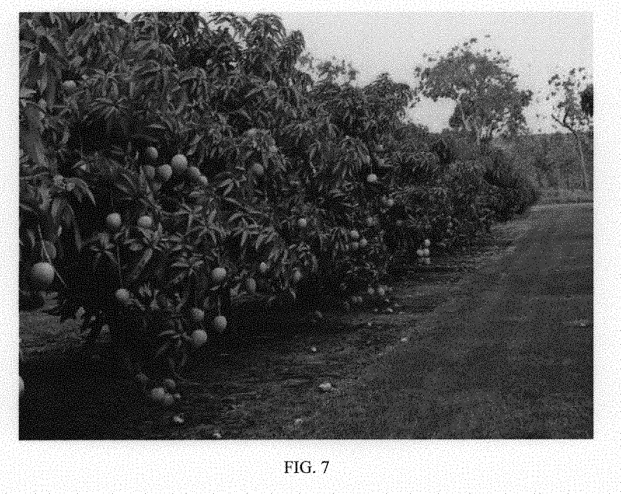

FIG. 8—illustrates a specimen of mature ripening fruit growing on a branch.

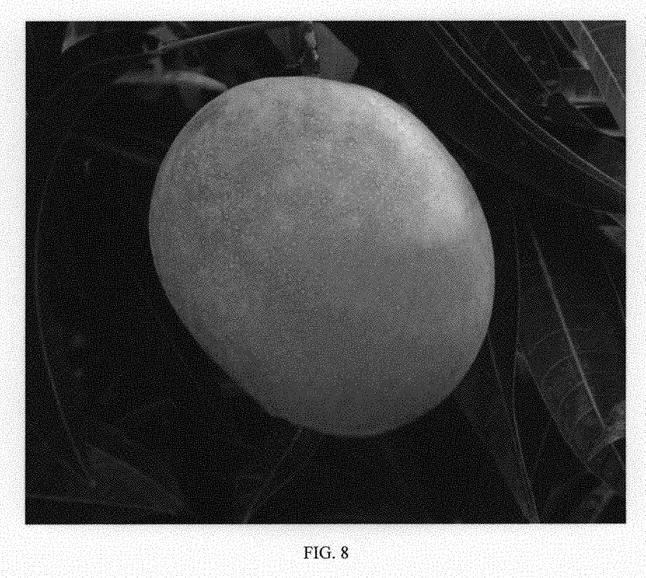

FIG. 9—illustrates a specimen of ripe fruit—halved—displaying flesh, halved endocarp, and halved embryo.

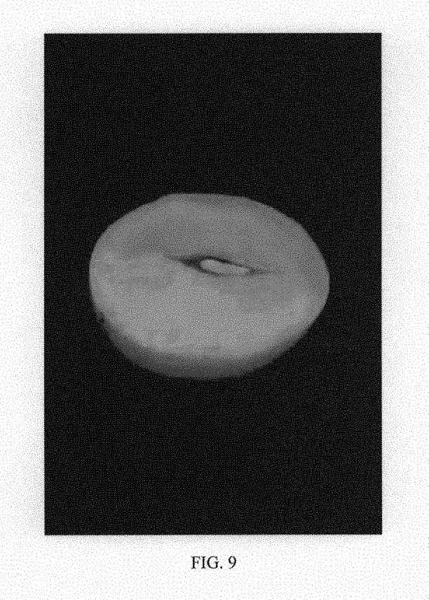

FIG. 10—illustrates a comparison of fruit from plants of the new variety and that of the ‘Kensington Pride’ variety.

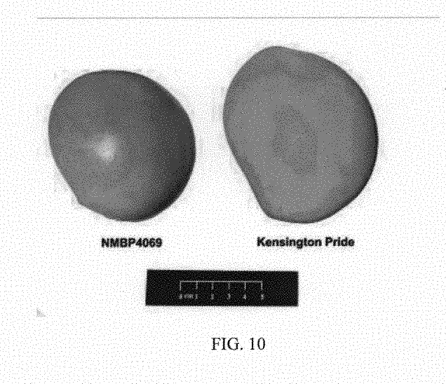

FIG. 11—illustrates specimens of fruit halved and whole and illustrates a specimen of the embryo and fibrous endocarp.

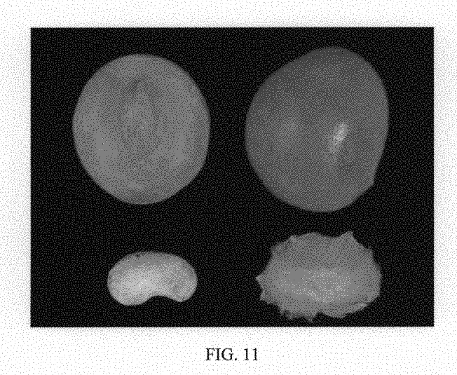

FIG. 12—illustrates specimens of ripe fruit.

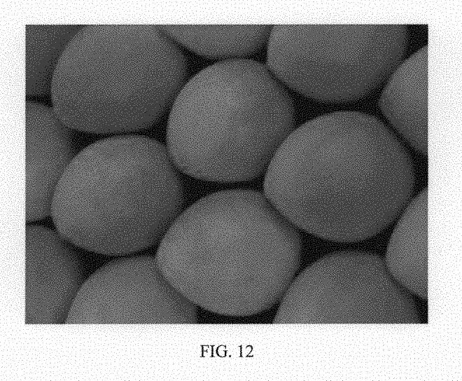

FIG. 13—illustrates a specimen of ripe fruit—side view.

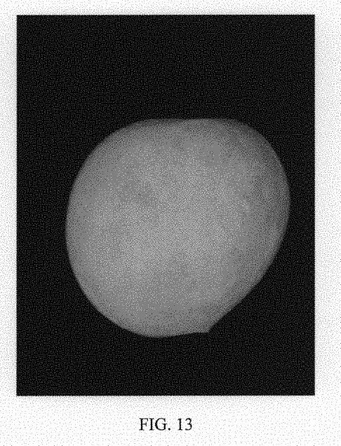

FIG. 14—illustrates a specimen of ripe fruit—opposite side view.

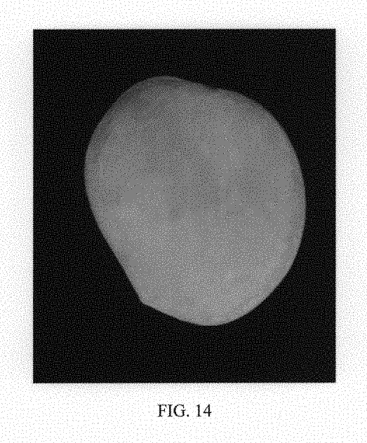

FIG. 15—illustrates a specimen of ripe fruit—top view—showing peduncle attachment.

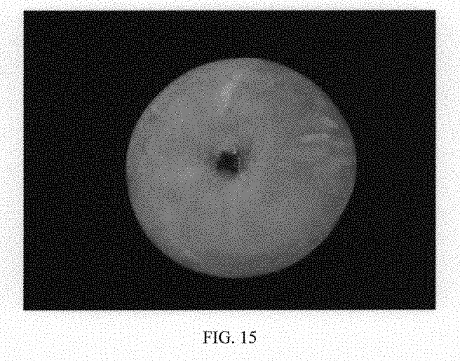

FIG. 16—illustrates a specimen of ripe fruit—halved—displaying flesh and intact fibrous endocarp.

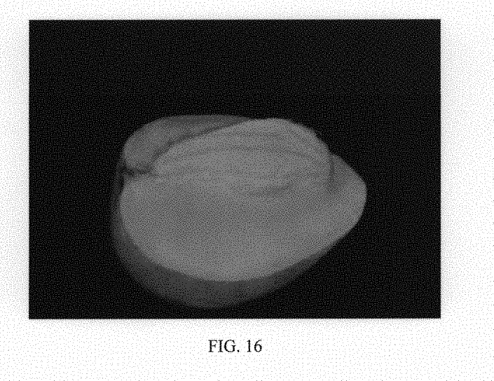

### DETAILED BOTANICAL DESCRIPTION

The chart used in the identification of colors is that of The Royal Horticultural Society (“R.H.S.” Colour Chart), London, England, Fourth Edition, 2001. The terminology which precedes reference to the chart has been added to indicate the corresponding color in more common terms. The description is based on a trial design which was a randomized complete block design with five single 8-10 year old tree replicates in Queensland, Australia. 10 to 20 random measurements of each character were assessed on each of the five single tree replicates.

### PLANT

a. Vigor: Commonly medium to high vigor tree with a compact medium to dense canopy, and the canopy shape is spreading.
b. Attitude of main branches: Erect to horizontal and spreading.
c. Height: Commonly 4 to 6 m in 8 to 10-year-old trees under standard annual pruning.
d. Width: Commonly 4 m in 8 to 10-year-old trees under standard annual pruning.
e. Bark: Commonly no striation and medium rough; texture is medium; and color is commonly near Grey-Brown Group 199B.

### LEAVES

a. Anthocyanin: Present with medium to strong intensity and a brownish coloration on immature leaves.
b. Leaf blade:
   Shape.—Generally elliptic with an obtuse base and the apex shape varies approximately 34% being attenuate, 40% being acuminate, and 26% being acute, straight in cross section, and is commonly always symmetric.
   Relief of upper face.—Smooth.
   Undulation of margin.—Weakly present.
   Attitude.—Horizontal.
   Size.—Length is typically 195.3 mm±8.94 std. dev. on average; width is typically 51.57 mm±2.13 std. dev. on average; and average length to width ratio is typically 3.84:1±0.16 std. dev.
   Color of lower surface of mature leaf.—Commonly near Yellow-Green Group 146B.
   Color of upper surface of mature leaf.—Commonly near Green Group 137A.
   Color of lower surface of immature leaf.—Commonly near Greyed-Orange Group 177A.
   Color of upper surface of immature leaf.—Commonly near Greyed-Orange Group 166A.
   Twisting.—Absent.
   Curvature of midrib.—Present and position is commonly from apex.
   Spacing of secondary veins.—Medium.
   Predominant relief of veins on upper surface.—Smooth.
   Serration.—Absent.
   Fragrance.—Present.
   Venation.—Pinnate with medium secondary veins which are approximately 11.33 mm±0.62 std. dev. apart, and the color is commonly near Green-Yellow Group 1B.
c. Petiole:
   Attitude.—Perpendicular.
   Length.—Very short to short, commonly between 10 and 33 mm, approximately 17.45 mm on average.
   Diameter.—Commonly between 2.32 and 3.58 mm.
   Color.—Not observed at this stage.

### INFLORESCENCE

a. Attitude of axis: Horizontal.
b. Size: Length is typically 302.5 mm±22.99 std. dev. on average; width is typically 176.25 mm±23.43 std. dev. on average; and length to width ration is typically 1.81:1±0.29 std. dev. on average.
c. Number of inflorescence branches: 20.66±2.04 std. dev., on average.
d. Color of axis and branches: Commonly near Red-Purple Group 58A.
e. Pubescence on axis and branches: Very sparse.
f. Leafy bracts: Present; size, shape and color designation not observed.
g. Number of flowers per inflorescence: Variable, commonly between 300 and 1500.
h. Size of flower: Diameter is approximately 7.35 mm±0.26 std. dev., on average.
i. Position of fertile stamen(s) in relation to style: Parallel.
j. Length of fertile stamen(s) in relation to style: Equal.
k. Development of staminodes: Weak to medium.
l. Anthocyanin coloration: Medium to strong intensity present in old flowers.
m. Inflorescence type: Narrowly to broadly conical panicle.
n. Percentage of hermaphrodite flowers and male flowers pre inflorescence: Varies depending on temperature during inflorescence development.
o. Color of petal pre-anthesis: Commonly near Green-Yellow Group 1D.
p. Color of sepal pre-anthesis: Commonly near Green-Yellow Group 145C.
q. Color of flower peduncle pre-anthesis: Commonly near Green-Yellow 145A.
r. Color of wilted petal post-anthesis: Commonly near Red-Purple Group 58C.
s. Color of sepal post-anthesis: Commonly near Green-Yellow Group 145A.
t. Color of flower peduncle post-anthesis: Commonly near Green-Yellow Group 145A.
u. Anthocyanin coloration wilted petal post-anthesis: Present, intensity is medium to strong.
v. Pistil: Single.
w. Staminodes: Typically 3 present.
x. Filament and style: Color not observed.

### FRUIT

a. Shape: Ovate and round with a slight beak; slightly sunken stem attachment; and both dorsal and ventral shoulders are rounded outward.
b. Flavor: Commonly a sweet rich ‘Kensington Pride’ variety style with a slight tang, no turpentine flavor.
c. Mature fruit:
   Size.—Length is approximately 98.43 mm±3.89 std. dev., on average, width is approximately 95.42 mm±5.78 std. dev., on average, depth is approximately 86.3 mm on average, and length/width ratio is approximately 1.03±0.06 std. dev., on average.
   Weight.—Approximately 378 g on average, commonly 325 to 500 g.
   Skin color.—Background is commonly near Yellow-Green Group 151A, blush is commonly near Red Group 44A over 50% of the skin when grown in the sun; fruit inside the canopy do not blush.
   Bloom on skin.—Inconspicuous.
   Lenticels.—Medium density with weak to medium conspicuousness; size is medium; color is commonly yellow; and absent is a roughness of the surface caused by the lenticels.
   Stalk cavity.—Present with a shallow depth.
   Neck.—Absent.
   Groove in ventral shoulder.—Present with a short length and very shallow to shallow depth.
   Lumpiness on ventral shoulder.—Absent.
   Sinus proximal of stylar scar.—Absent.
   Bulge proximal of stylar scar.—Absent.
   Shape at stylar scar.—Pointed.
d. Ripe fruit:
   Skin color.—Predominantly yellow and red, background is commonly near Yellow-Orange Group 22B, and blush is commonly near Red Group 44A with a medium degree of speckled pattern of skin color.
   Thickness of skin.—Medium to thick; approximately 0.86 mm on average.
   Adherence of skin to flesh.—Weak to medium.
   Main color of flesh.—Commonly near Yellow-Orange Group 23A.
   Firmness.—Soft; approximately 1.02 mm depression, 50 g for 30 sec on average, similar to the ‘R2E2’ variety.
   Juiciness.—Medium.
   Texture of flesh.—Medium.
   Pulp color.—Commonly yellow/orange.
   Pulp texture.—Commonly soft.
   Pulp fiber.—Very low.
   Pulp depth.—Approximately 30 mm on average in a cut cheek.
e. Fruit count per panicle: Predominantly single fruit per panicle.
f. Seed:
   Prominence of point at stylar area.—Weak to medium.
   Relief of stone surface.—Smooth to grooved.
   Sharp points on surface.—Absent.
   Fibres on cheeks of stone.—Short and medium density.
   Texture of fibre.—Fine.
   Endocarp.—Medium to thick thickness, color is not observed.

### DEVELOPMENT

a. Seed embryony: Monoembryonic.
b. Time of fruit maturity: Medium, varies with temperature; in Mareeba district of Queensland Australia the typical length of fruit maturing is between three months and four months, one week.
c. Cropping: Commonly seven-year-old trees in their fourth year of cropping produce approximately 61 to 171 kg of fruit per tree.
d. Harvest season: Commonly early to mid-season.
e. Yield: Commonly medium to heavy with tendency for biennial bearing.
f. Hardiness: Coldest observed temperature withstood was 4° C.; hottest observed temperature withstood was 39° C.
g. Bloom: Varies from year to year and typically between mid-July and late September in Mareeba district of Queensland Australia.
h. Pest/disease resistance: Not observed to date.
i. Shipping quality, fruit storage life, fruit market use: Not observed to date.

Plants of the ‘NMBP-4069’ variety have not been observed under all possible environmental conditions to date. Accordingly, it is possible that the phenotypic expression may vary somewhat with changes in light intensity and duration, cultural practices, and other environmental conditions.

I claim:

1. A new and distinct Mango plant characterized by the following combination of characteristics:
(a) displays a medium vigor plant with a compact dense canopy,
(b) exhibits a medium to heavy yielding with a tendency for biennial bearing,
(c) produces monoembryonic seed,
(d) has a harvest season that is early to mid-season, and
(e) provides a fruit with a sweet rich ‘Kensington Pride’ variety style flavor with a slight tang;
substantially as herein shown and described.
